(19) United States
(12) Patent Application Publication
(10) Pub. No.: US 2012/0203160 A1
(43) Pub. Date: Aug. 9, 2012

(54) IMPLANTS FOR REDUCING INTRAOCULAR PRESSURE

(75) Inventors: Malik Y. Kahook, Denver, CO (US); Naresh Mandava, Denver, CO (US); Robin Shandas, Boulder, CO (US); Bryan Rech, Boulder, CO (US)

(73) Assignee: THE REGENTS OF THE UNIVERSITY OF COLORADO, A BODY CORPORATE, Denver, CO (US)

(21) Appl. No.: 13/501,599

(22) PCT Filed: Oct. 12, 2010
(86) PCT No.: PCT/US10/52350

§ 371 Date: Apr. 12, 2012

Related U.S. Application Data
(60) Provisional application No. 61/250,815, filed on Oct. 12, 2009.

(51) Int. Cl. A61F 9/007

(52) U.S. Cl. 604/8

(57) ABSTRACT

The present invention provides ocular implants adapted to reside in Schlemm's canal for reducing intraocular pressure of an eye and methods for using the same. In some embodiments the ocular implants comprise a thin rod adapted and configured to extend in a curved volume in Schlemm's canal. The thin rod comprises a plurality of wave-shaped segments such that a sufficient number and amount of wave-shaped segments extend to the inner wall of the trabecular meshwork and to the outer wall of Schlemm's canal thereby keeping Schlemm's canal open.

### CROSS-REFERENCE TO RELATED APPLICATIONS

This application claims the priority benefit of U.S. Provisional Application No. 61/250,815, filed Oct. 12, 2009, which is incorporated herein by reference in its entirety.

### FIELD OF THE INVENTION

The present invention relates generally to devices that are implanted within the eye. More particularly, the present invention relates to devices that reduces intraocular pressure of an eye.

### BACKGROUND OF THE INVENTION

Glaucoma is the leading cause of irreversible blindness worldwide and the second leading cause of blindness, behind cataract. While many glaucoma risk factors such as family history of glaucoma, advanced age, and race (African or Latino) have been identified, increased intraocular pressure is the only known risk factor modifiable by medical or surgical intervention. Glaucoma is a progressively degenerative condition affecting millions of people worldwide. More than 50% of patients with glaucoma will eventually require a laser or surgical intervention to lower intraocular pressure. Lowering of intraocular pressure has been shown to slow progression of visual field loss in ocular hypertensive patients as well as in various forms of glaucoma.

The eye can be conceptualized as a ball filled with fluid. There are two types of fluid inside the eye. The cavity behind the lens is filled with a viscous fluid known as vitreous humor. The cavities in front of the lens are filled with a fluid known as aqueous humor. Whenever a person views an object, the object is viewed through both the vitreous humor and the aqueous humor. In addition, the object is also viewed through the cornea and the lens of the eye. The cornea and the lens are transparent, and there are no blood vessels within these tissues. Therefore, no blood flows through the cornea and the lens to provide nutrition to these tissues and to remove wastes from these tissues. These functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the anterior chamber of the eye through the trabecular meshwork and into Schlemm's canal as new aqueous humor is secreted by the epithelial cells of the ciliary body. This aqueous humor enters the venous blood stream from Schlemm's canal and is carried along with the venous blood leaving the eye.

When the natural drainage mechanisms of the eye stop functioning properly, the pressure inside the eye begins to rise. Researchers have shown that prolonged exposure to high intraocular pressure causes damage to the optic nerve that transmits sensory information from the eye to the brain. This damage to the optic nerve results in loss of peripheral vision. As glaucoma progresses, more and more of the visual field is lost until the patient is completely blind.

Currently, several classes of medications exist for both topical and oral treatment of elevated intraocular pressure. Most of these medications, with the exception of prostaglandin analogs, decrease aqueous humor production rather than targeting the fluid outflow tissue (Trabecular Meshwork) commonly believed to be the primary site of dysfunction in open angle glaucoma. When medications fail, ophthalmologists often resort to treating the trabecular meshwork with lasers that increase fluid outflow from the eye. The effect of laser treatment is unfortunately often short lived and many patients do not respond at all to this mode of therapy. Invasive filtration surgery, allowing for efflux of fluid out of the eye to decrease intraocular pressure, is the procedure of choice once both medications and laser have failed. Filtration surgery is often successful in the early stages at decreasing intraocular pressure, but carries with it a relatively high rate of failure, i.e., about 50% failure within 5 years. Filtration surgery also exposes the eye to multiple complications such as endophthalmitis (infection of the eye with loss of vision), pain, double vision and cosmetically undesirable whitening of the tissue around the iris. The surgery is also complex and requires a great deal of expertise. Another method is installing a glaucoma drainage device (e.g., silicone tube connected to a silicone plate that is implanted beneath the conjunctiva). This method, however, does not result in intraocular pressure lowering equivalent to trabeculectomy and still carries with it the risk of infection and loss of vision.

Therefore, for at least these reasons, there is a need for an alternative surgical procedure that is minimally invasive, more easily reproducible, and free of serious side effects for patients suffering from increased intraocular pressure or glaucomatous optic neuropathy.

### SUMMARY OF THE INVENTION

One aspect of the invention provides ocular implants that are designed to be inserted into Schlemm's canal of an eye to facilitate the flow of aqueous humor out of the anterior chamber of the eye by, e.g., supporting tissue in the trabecular meshwork and in Schlemm's canal. Generally, the ocular implants of the invention are adapted and configured to reside completely within Schlemm's canal of an eye. When implanted ocular implants of the invention conform to, or support, the inner lumen of Schlemm's canal. Ocular implants of the invention are designed to extend to the inner wall of the trabecular meshwork and to the outer wall of Schlemm's canal to keep Schlemm's canal open. By supporting the inner lumen structure of Schlemm's canal, ocular implants of the invention prevent collapse of inner lumen of Schlemm's canal and reduce intraocular pressure, thereby reducing the risk of glaucoma.

Typically, ocular implants of the invention comprise a thin rod adapted and configured to extend in a curved volume whose longitudinal axis defines a plane when the rod resides in Schlemm's canal of the eye. The thin rod comprises a plurality of non-linear (relative to longitudinal axis of the rod), e.g., wave-shaped segments, a diameter in the range of about 5 to about 400 μm, a total length in the range of from about 0.5 to about 40 mm, a sufficient amount of tensile strength, and a sufficient number and amount of wave-shaped segments that extend to the outer wall of the trabecular meshwork and to the outer wall of Schlemm's canal to keep Schlemm's canal open.

The ocular implant of the invention facilitates flow by maintaining the structure (i.e., opening) of Schlemm's canal. By keeping Schlemm's canal open the ocular implant of the invention allows aqueous humor to flow axially along Schlemm's canal, into Schlemm's canal from the anterior chamber of the eye, and leaving Schlemm's canal via the outlets that communicate with the canal. Without being bound by any theory, it is believed that after exiting Schlemm's canal via the outlets, aqueous humor enters the venous blood stream and is carried along with the venous blood leaving the eye. The pressure of the venous system is typically around 5-10 mmHg above atmospheric pressure. Accordingly, the venous system provides a pressure backstop which assures that the pressure in the anterior chamber of the eye remains above atmospheric pressure.

Some ocular implants of the invention comprise a thin rod that conforms to the inner lumen of Schlemm's canal in such a manner as to support inner lumen of Schlemm's canal by extending at least some portions of the rod to the outer wall of the trabecular meshwork and at least some portion of the rod to the outer wall of Schlemm's canal. Such a support of inner lumen of Schlemm's canal keeps Schlemm's canal open to allow flow of aqueous humor. As should be appreciated, the thin rod has a sufficient amount of tensile strength to maintain opening of Schlemm's canal, thereby maintaining fluid communication between the anterior chamber and the collection channels of the eye. In some embodiments, ocular implants of the invention utilize advantage of the unique properties of shape memory polymers to allow insertion of the ocular implants through needles or canula in a linear form and are reshaped into a desired form once placed in Schlemm's canal by thermal actuation of the shape memory effect. In some embodiments, ocular implants of the invention bypass the major cause of complication in current invasive techniques for the treatment of glaucoma (e.g., creation of a fistula which has a 50% failure rate) and allow the natural drainage system to function more appropriately to reduce intra-ocular pressure.

### BRIEF DESCRIPTION OF THE DRAWINGS

FIG. 1 is a plan view showing a portion of an eye.

FIG. 2 is a schematic illustration of one embodiment of an ocular implant of the present invention.

FIGS. 3A and 3B are schematic illustrations of two different embodiments of the ocular implants of the present invention placed in Schlemm's canal.

FIG. 4 is an illustration of another ocular implant embodiment of the present invention.

### DETAILED DESCRIPTION OF THE INVENTION

Some aspects of the invention provide ocular implants that maintain fluid communication between the anterior chamber and the collection channels of the eye by keeping Schlemm's canal open. In some embodiments, ocular implants of the invention take advantage of the unique properties of shape memory polymers to allow insertion of the ocular implants through needles or canula in a linear form. Once placed in Schlemm's canal, ocular implants are reshaped into a desired form by thermal actuation of the shape memory effect.

Several advantages are offered by ocular implants of the present invention. For example, ocular implants of the invention reduce the risk of major cause of complication in current invasive techniques (e.g., creation of a fistula) and allow the natural drainage system to function more appropriately in preventing glaucoma. In addition, ocular implants of the invention are typically implanted through a small incision in the cornea, similar to techniques used in cataract surgery that has a great safety profile. Moreover, the ocular implant implantation generally takes place inside the eye with no portion of the ocular implant exiting the eye chamber. Such implantation also leads to a lower chance of infection. Another significant advantage of ocular implants of the present invention is that the skill level involved in implantation is much lower than that required to perform either trabeculectomy or glaucoma drainage device implantation. Thus, more physicians can perform this procedure leading to greater patient access.

Ocular implants of the invention comprise a thin rod having a diameter ranging from about 5 to about 400 microns, typically from about 30 to about 300 microns, and often from about 100 to about 200 microns. The total length of the thin rod is from 0.5 mm to about 40 mm, typically from about 1 mm to about 20 mm, and more often from about 2 mm to about 12 mm. By using a shape memory polymer in some instances, the pre-deployed or “stored” shape of the ocular implants is minimized to reduce the incision size for entry and delivery via cannula or a needle. The ocular implants are then “activated” or deployed through a temperature stimulus to expand and conform to the inner lumen of Schlemm's canal and extend into the trabecular meshwork and to the anterior chamber of the eye. In this way ocular implants of the invention serve at least two therapeutic functions: (i) keeping the Schlemm's canal open under various intraocular pressures; and (ii) placing the trabecular meshwork beams in stretched form to prevent collapse of Schlemm's canal or alterations in the architecture, thereby avoiding occlusion of aqueous filtrating in to the Schlemm's canal.

In order to prevent collapse or alterations in the Schlemm's canal, ocular implants of the invention have a sufficient tensile strength to prevent collapse of inner lumen of Schlemm's canal. In some embodiments, the tensile strength of ocular implants of the invention is at least about 5 psi, often at least about 10 psi and more often at least about 100 psi. Alternatively, the tensile strength of a typical ocular implant of the invention ranges from about 5 psi to about 5,000 psi, typically from about 5 psi to about 2,000 psi, and often from about 5 psi to about 1,000 psi. However, it should be appreciated that the scope of the invention is not limited to these particular tensile strengths.

The present invention will be described with regard to the accompanying drawings which assist in illustrating various features of the invention. It should also be noted that like elements in different drawings are numbered identically. The present invention generally relates to ocular implants for lowering intraocular pressure. It should be appreciated that the drawings, which are not necessarily to scale, depict exemplary embodiments and are not intended to limit the scope of the invention. Examples of constructions, materials, dimensions, and manufacturing processes are provided for selected elements. All other elements employ that which is known to those of skill in the field of the invention. Those skilled in the art will recognize that many of the examples provided have suitable alternatives that can be utilized.

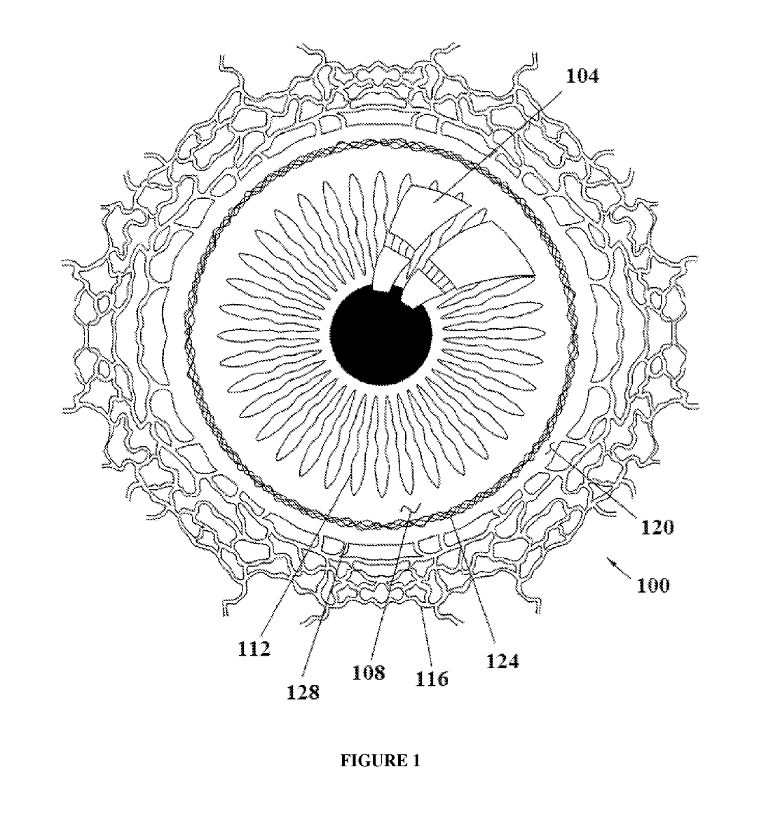

FIG. 1 is a plan view showing a portion of an eye 100. A reflection on the outer surface of the cornea 104 of eye 100 is visible in this Figure. Cornea 104 encloses an anterior chamber 108 of eye 100. The iris 112 of eye 100 is visible through cornea 104 and anterior chamber 108. Anterior chamber 108 is filled with aqueous humor which helps maintain the generally hemispherical shape of cornea 104. When a person views an object, the light is transmitted through the cornea, the aqueous humor, and the lens of the eye. The cornea and lens must be transparent to avoid distorting the vision. For at least this reason, the cornea and the lens cannot have any blood vessels. This lack of blood vessels also means that no blood flows through the cornea and the lens to provide nutrients and to remove wastes from these tissues. These functions are performed by the aqueous humor. A continuous flow of aqueous humor through the eye provides nutrition to portions of the eye (e.g., the cornea and the lens) that have no blood vessels and removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye. The structures that drain aqueous humor from anterior chamber 108 include Schlemm's canal 120 and a large number of veins 116.

In FIG. 1, Schlemm's canal 120 can be seen encircling iris 112. Aqueous humor exits anterior chamber 108 and enters Schlemm's canal 120 by flowing through a trabecular mesh 124. Aqueous humor exits Schlemm's canal 120 by flowing through a number of outlets 128. After leaving Schlemm's canal 120, aqueous humor travels through veins 116 and is absorbed into the blood stream. Schlemm's canal typically has a non-circular cross-sectional shape whose diameter can vary along the canal's length and according to the angle at which the diameter is measured. In addition, there may be multiple partial pockets or partial compartments (not shown in these figures) formed along the length of Schlemm's canal. The shape and diameter of portions of Schlemm's canal and the existence and relative location of partial pockets or compartments may limit or prevent fluid flow from one point of Schlemm's canal to another. Hence, each outlet 128 from Schlemm's canal may drain only a portion of Schlemm's canal. It will be appreciated that a number of outlets 128 communicate with Schlemm's canal 120. After leaving Schlemm's canal 120, aqueous humor travels through veins 116 and is absorbed into the blood stream.

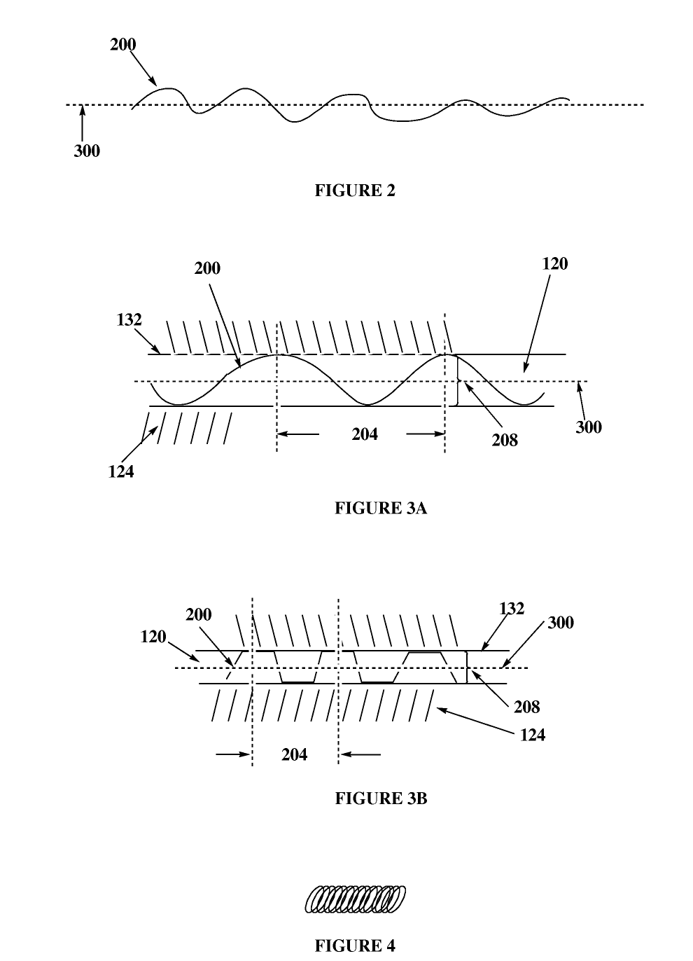

FIG. 2 shows one embodiment of the ocular implant of the invention 200. As can be seen, in this embodiment, the ocular implant is a thin rod 200 adapted and configured to extend in a curved volume whose longitudinal axis 300 defines a plane when the thin rod is inserted in Schlemm's canal. When placed in Schlemm's canal, implant conforms to the inner lumen of Schlemm's canal as illustrated schematically in FIGS. 3A and 3B. Moreover, there is a sufficient number of non-linear (i.e., wave-shaped) segments that extend to the outer wall of the trabecular meshwork 124 and to the outer wall 132 of Schlemm's canal to keep Schlemm's canal open. By supporting the inner lumen structure of Schlemm's canal, ocular implant 200 facilitates the outflow of aqueous humor from the anterior chamber 108. This flow can include axial flow along Schlemm's canal, flow from the anterior chamber into Schlemm's canal, and flow leaving Schlemm's canal via outlets communicating with Schlemm's canal. When in place within the eye, ocular implant 200 has shown to reduce intraocular pressure. Ocular implant 200 includes a plurality of wave-shaped segments such that the sufficient amount of inner lumen of Schlemm's canal is supported to reduce intraocular pressure. As used herein, the term “wave-shaped segment” refers to any configuration in which a portion of the thin rod is not in the longitudinal axis 300. Such a wave-shaped segment can be sinusoidal, trapezoidal, rectangular, or any other non-linear shape or a combination thereof. In addition, as shown in FIG. 4, the thin rod can also be in a helical, or a tubular configuration.

Referring again to FIGS. 3A and 3B, the ocular implant 200 comprises a plurality of wave-shaped segments that extend to the inner wall of the trabecular meshwork 124 and to the outer wall 132 of Schlemm's canal to keep Schlemm's canal open. Each wave-shaped segment can be independently shaped and configured. In general, the maximum wave length 204 of the wave-shaped segments of the ocular implant 200 is about 10 mm or less, typically 5 mm or less, and often 3 mm or less. It should be appreciated that the smaller wave length provides more contact with the trabecular meshwork 124 and the outer wall 132 of Schlemm's canal and generally provides more support to the inner lumen of Schlemm's canal.

As stated above, Schlemm's canal typically has a non-circular cross-sectional shape whose diameter can vary along the canal's length and according to the angle at which the diameter is measured. Thus, the wave amplitude 208 of ocular implant 200 need not be consistent throughout the plurality of wave-shaped segments. While the wave amplitude 208 can vary within each wave-shaped segment, generally the maximum wave amplitude 208 is about 2 mm or less, typically 1 mm or less, and often about 0.5 mm or less.

It should be appreciated that the ocular implant 200 does not need to inserted along the entire length of Schlemm's canal. In fact, in some instances the ocular implant 200 is inserted in only a portion of the entire length of Schlemm's canal, for example, about 80% or less, typically about 60% or less, and often about 50% or less of the total length of Schlemm's canal. In other embodiments, a plurality of ocular implants 200 can be inserted in various areas of Schlemm's canal.

Ocular implants can be made from a wide variety of materials including, but not limited to, a material comprising a shape-memory material such as a shape-memory polymer. Typically, the shape-memory polymer is non-wave shaped at room temperature. This non-wave shaped configuration allows ease of insertion in to Schlemm's canal. Once inserted into Schlemm's canal, these shape-memory polymers are “activated” or “reconfigured” to a plurality of wave-shaped segments by thermal activation, i.e., temperature within Schlemm's canal compared to room temperature. Alternatively, ocular implant 200 can be fabricated with a plurality of wave-shaped segments and inserted into Schlemm's canal.

A wide variety of materials can be used to produce ocular implants of the invention including, but not limited to, a biocompatible polymer, medical grade stainless steel, titanium, nitinol, or plastic, metallic, glass, polyether ether ketone, thermoplastic materials, thermal set materials, photosensitive plastics, and acrylic materials. The biocompatible polymer can include, but not limited to, hydrogels, which are well known to one skilled in the art, acrylate, methacrylate, and a mixture thereof.

Shape-memory materials are materials that, after deformation, are able to recover their initial shape upon the action of a stimulus. These materials have found numerous applications as implantable biomedical devices, particularly as stents, as the capacity for collapsing an otherwise unwieldy device and returning it to its original shape in situ enables minimally-invasive delivery approaches for device implantation. Shape-memory polymers are particularly attractive for biomedical applications as their mechanical properties can be adjusted to match the tissue of the implant site. Moreover, implanted polymeric devices can act as convenient drug-delivery vehicles as therapeutic agents are readily incorporation in polymeric matrices.

There are a number of shape-memory materials, including polymer formulations, ceramics, metals, etc., suitable for implantable medical devices. See, for example, Lendlein et al. in Angew. Chem. Int. Ed., 2002, 41, 2034-2057, which is incorporated herein in its entirety. Shape memory polymers can be created from various formulations of polymers, including natural and synthetic polymers. Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(β-hydroxybutyrate), poly(3-hydroxyoctanoate) and poly(3-hydroxyfatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of such polymers include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt, all collectively referred herein as celluloses.

Representative synthetic degradable polymer segments include polyhydroxy acids, such as polylactides, polyglycolides and copolymers thereof; poly(ethylene terephthalate); polyanhydrides, poly(hydroxybutyric acid); poly(hydroxyvaleric acid); poly[lactide-co-(.epsilon.-caprolactone)]; poly[glycolide-co-(.epsilon.-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Their hydrolytic degradation rates can generally be altered by simple changes in the polymer backbone and their sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof.

The polymers can be obtained from commercial sources such as Sigma Chemical Co. (St. Louis, Mo.); Polysciences (Warrenton, Pa.); Aldrich Chemical Co. (Milwaukee, Wis.); Fluka (Ronkonkoma, N.Y.); and BioRad (Richmond, Calif.). Alternately, the polymers can be synthesized from monomers obtained from commercial sources, using standard techniques.

In one embodiment, the SMPs can be photopolymerized from tert-butyl acrylate (tBA) di-functional monomer with polyethylene glycol dimethacrylate (PEGDMA) tetra-functional monomer acting as a crosslinker. A di-functional monomer may be any compound having a discrete chemical formula further comprising an acrylate functional group that will form linear chains. A tetra-functional monomer may be any compound comprising two acrylate, or two methacrylate groups. A crosslinker may be any compound comprising two or more acrylate or methacrylate functional groups. Also, ethyleneglycol, diethyleneglycol, and triethyleneglycol based acrylates are forms of polyethyleneglycol based acrylates with only one, two, or three repeat units.

In another embodiment, the SMPs may be photopolymerized from three or more monomers and/or homopolymers to achieve a range of desired thermomechanical properties. An SMP formed from three or more monomers and/or homopolymers may achieve a much larger range of rubbery modulus to glass transition temperature, rather than that obtained from a strictly linear combination between two monomers or homopolymers. For example, a combination of tert-butyl acrylate (tBA), polyethylene glycol dimethacrylate (PEGDMA), and diethyleneglycol dimethacrylate (DEGDMA), may be employed in SMP photopolymerization.

In one aspect, the amount of crosslinker used in SMP polymerization is greater than about 10%. In another aspect, the SMP is designed to have modulus values between 1 and 50 MPa. In a further aspect, the deployment time may be varied from about 5 seconds to about 800 seconds.

In a further embodiment, the SMP material is a photo-initiated network comprising of tert-butyl acrylate (tBA), polyethyleneglycol dimethacrylate (PEGDMA), and 2,2-dimethoxy-2-phenylacetephenone as a photo-initiator. In a one embodiment, controlling the amount of cross-linking PEGDMA, the glass transition temperature (T_{g}) was tailored to from about 25° C. to about 55° C., which makes the polymer optimal for shape recovery at body temperature. Other polymerization techniques, such as thermal radical initiation, can be used for polymer fabrication.

Specific areas of interest for SMP include mechanical properties (e.g., tensile strength), transition temperature, transition rate, shape fixity, etc. Such properties can be adjusted, for example, by the amount of cross-linking as well as selection of polymeric materials. In fact, shape memory polymers with different properties are well known to one skilled in the art. See, for example, U.S. patent application Ser. No. 12/295,594, filed Mar. 30, 2006, and U.S. Provisional Patent Application No. 61/047,026 entitled “Thiol-Vinyl Systems for Shape Memory Polymers,” the disclosures of which are incorporated herein in their entirety.

Typically, ocular implants of the invention require dimensional precision that is significantly greater than those that can be made by conventional UV tooling/mold materials. Thus, shape-memory polymer materials that are used to produce ocular implants of the invention are formulated with a suitable catalyst to provide for thermal polymerization. Such methods of production allow the precision molds to be fabricated from steel, aluminum or other traditional injection mold materials. Suitable catalysts for thermopolymerization are well known to one skilled in the art and include benzoyl peroxide. Some of the characteristics of shape-memory polymer precursors include materials with desired post cured mechanical properties while maintaining a sufficiently low viscosity for mold filling. Ocular implants can be evaluated for shape recovery repeatability (i.e., shape certainty), for example, via heating, compressing to the defined stored shape, cooling, reheating and recovery. Results are evaluated to identify key sensitivities to the design, formulation and process. The formulation and process are iterated as needed to achieve suitable dimensional repeatability for desirable properties.

Another aspect of the invention provide computer aided design (CAD) of Schlemm's canal that can be used for testing various ocular implant devices. One of the tests is to evaluate the ability of an ocular implant to properly enter this anatomical feature through a suitable cannula size, and then deploy to form a clinically effective interface with Schlemm's canal and the trabecular meshwork structure. Other areas of using CAD are to provide a simulation of conduit for fluid communication between the anterior and exterior chambers of the eye using the ocular implant and to observe the effect of placement of the ocular implant on trabecular meshwork in tension (or stretch). Dimensional position and contact are evaluated under digital microscopy with measurement recording.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

A human eye perfusion model (i.e., a cadaver eye) was used to investigate intraocular pressure lowering by ocular implants of the present invention. The ocular implant was made by micromolding process. Stead state intraocular pressure was achieved using a constant flow set-up with Dulbecco's fluid. Baseline intraocular pressure values were recorded. An ocular implant measuring 150 microns in diameter, 8 mm in length, and having a maximum wave length of 1 mm and maximum amplitude of 400 microns was then threaded through Schlemm's canal and the surgical site was sealed using 10-0 nylon suture and cyanoacrylate glue until the wound was water tight. The perfusion set-up was then re-initiated and the new steady state intraocular pressure was recorded using pneumatonometry and an indwelling pressure gauge. This was repeated 4 times in human eyes free of glaucoma. All eyes were pseudophakic. Intraocular pressure values were as follows:

### Intraocular Pressure

| | Pre-implant steady state | Post-implant steady state |
|---|---|---|
| 1. | 16 mm Hg | 9 mm Hg |
| 2. | 18 mm Hg | 14 mm Hg |
| 3. | 20 mm Hg | 14 mm Hg |
| 4. | 16 mm Hg | 11 mm Hg |

As the results indicate, all eyes experienced a significant decrease in intraocular pressure with ocular implants of the present invention.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An ocular implant adapted and configured to reside completely within Schlemm's canal of an eye, wherein when implanted within Schlemm's canal said implant conforms to the inner lumen of Schlemm's canal and comprises a thin rod adapted and configured to extend in a curved volume whose longitudinal axis defines a plane when said rod resides in Schlemm's canal of the eye and wherein said thin rod comprises a plurality of wave-shaped segments, a diameter in the range of about 5 to about 400 μm, a total length in the range of from about 0.5 to about 40 mm, a sufficient amount of tensile strength, and a sufficient number and amount of wave-shaped segments that extend to the outer wall of the trabecular meshwork and to the outer wall of Schlemm's canal to keep Schlemm's canal open.

2. The ocular implant of claim 1, wherein the maximum wave length of the wave-shaped segments of said thin rod is about 5 mm or less.

3. The ocular implant of claim 1, wherein the maximum wave amplitude of the wave-shaped segments is at about 1 mm or less.

4. The ocular implant of claim 1, wherein the minimum tensile strength of the wave-shaped segment is at least about 5 psi.

5. The ocular implant of claim 1, wherein the diameter of said rod is from about 100 to about 200 μm.

6. The ocular implant of claim 1, wherein the total length of said thin-rod ranges from about 2 to about 12 mm.

7. The ocular implant of claim 1, wherein said thin-rod is made from a material comprising a shape-memory polymer.

8. The ocular implant of claim 7, wherein said shape-memory polymer is non-wave shaped at room temperature.

9. The ocular implant of claim 8, wherein said shape-memory polymer comprises a plurality of wave-shaped segments when placed within Schlemm's canal of an eye.

10. The ocular implant of claim 1, wherein said thin-rod is made from a material comprising a biocompatible polymer, medical grade stainless steel, titanium, nitinol, or plastic, metallic, glass, polyether ether ketone, thermoplastic materials, thermal set materials, photosensitive plastics or acrylic materials.

11. The ocular implant of claim 10, wherein the biocompatible polymer comprises acrylate, methacrylate, or a mixture thereof.

12. A method for reducing intraocular pressure in a subject, said method comprising inserting an ocular implant of claim 1 in Schlemm's canal of said subject.

13. The method of claim 12, wherein the ocular implant is implanted using device comprising a cannula or an injection device.

14. The method of claim 12, wherein said method reduces at least 1 mmHg of intraocular pressure.

15. The method of claim 12, wherein intraocular pressure is reduced by at least 10%.

16. A method for treating glaucoma in a subject, said method comprising inserting an ocular implant of claim 1 in Schlemm's canal of the subject in need of such a treatment.
